(19) United States
(12) Patent Application Publication
(10) Pub. No.: US 2026/0248166 A1
(43) Pub. Date: Aug. 27, 2026

(54) POLYELECTROLYTE COMPLEXES OF PROTEINS AND USES THEREOF

(71) Applicant: Firmenich SA, Satigny (CH)

(72) Inventors: Valeria LARCINESE-HAFNER, Satigny (CH); Philipp ERNI, Satigny (CH); Amal ELABBADI, Satigny (CH)

(73) Assignee: Firmenich SA, Satigny (CH)

(21) Appl. No.: 18/872,556

(22) PCT Filed: Jun. 13, 2023
(86) PCT No.: PCT/EP2023/065774

§ 371 Date: Dec. 6, 2024

Related U.S. Application Data
(60) Provisional application No. 63/352,016, filed on Jun. 14, 2022.

(30) Foreign Application Priority Data
Jun. 22, 2022 (EP) 22180320.8

(51) Int. Cl. A23L 27/00; A23C 11/10; A23J 3/14; A23J 3/18; A23J 3/20; A23J 3/22; C07K 19/00

(52) U.S. Cl. CPC A23L 27/84; A23C 11/103; A23J 3/14; A23J 3/18; A23J 3/20; A23J 3/227; C07K 19/00

(57) ABSTRACT

The present disclosure relates generally to polyelectrolyte complexes of non-animal proteins and their use in various comestible products, such as food products, beverage products, and nutritional products, including meat and dairy analogue products. In some embodiments, the polyelectrolyte complexes comprise a plant protein, such as pea protein, soy protein, canola protein, potato protein, and bean proteins, such as fava bean protein. In some embodiments, the polyelectrolyte complexes mask certain undesirable tastes of one or more of the proteins and improve the mouthfeel of comestible compositions that include the protein complexes. In some embodiments, the polyelectrolyte complexes are combined with certain fibers, which allows the blend to be suitable for addition to an ingestible composition useful for making meat or dairy analogue products. In some embodiments, flavorings or flavor-modifiers are combined with the polyelectrolyte complexes as well.

### TECHNICAL FIELD

The present disclosure relates generally to polyelectrolyte complexes of non-animal proteins and their use in various comestible products, such as food products, beverage products, and nutritional products, including meat and dairy analogue products. In some embodiments, the polyelectrolyte complexes comprise a plant protein, such as pea protein, soy protein, canola protein, potato protein, and bean proteins, such as fava bean protein. In some embodiments, the polyelectrolyte complexes mask certain undesirable tastes of one or more of the proteins and improve the mouthfeel of comestible compositions that include the protein complexes. In some embodiments, the polyelectrolyte complexes are combined with certain fibers, which allows the blend to be suitable for addition to an ingestible composition useful for making meat or dairy analogue products. In some embodiments, flavorings or flavor-modifiers are combined with the polyelectrolyte complexes as well.

### DESCRIPTION OF RELATED ART

The human diet generally includes both animal-derived and non-animal-derived products. In recent years, the proportion of calories consumed from animal-derived products has increased. This poses certain health-related concerns, as eating too much meat, especially meats high in fat and cholesterol, tends to contribute to heart disease and related problems. Another concern relates to sustainability. Raising animals for meat often requires large amounts of grain or grass to use as animal feed. It requires many times more acres of land to grow the grass or grain to feed such animals than it would to grow plants for direct human consumption. Thus, as the global population continues to increase, the demand for increasing agricultural space becomes steadily unsustainable.

Thus, there is increasing demand to replace animal-derived foods in the human diet with similar materials derived from plants, algae, fungi, and the like. In many cases, because consumers have become accustomed to consuming animal-derived foods, these alternative non-animal-based foods are designed to simulate the flavor, texture, and culinary experience of consuming animal-derived foods. Such non-animal-based foods are commonly referred to as meat analogues or dairy analogues. But creating such meat and dairy analogue materials poses a number of challenges, especially as one attempts to use plant-derived materials to create a food product that simulates meat and dairy products.

One such challenge involves masking certain undesirable tastes and improving the mouthfeel of plant-derived proteins. For example, pea protein is often used to make plant-based alternatives to meat and dairy products. But pea protein can impart undesirable vegetal and cereal tastes and is perceived as chalky by many consumers. Combining the plant protein with certain masking agents can help to remedy this problem to an extent. But these solutions must often be tailored to a particular protein or batches of a protein.

Therefore, there is a continuing need to develop a more general approach to masking off notes or improving the mouthfeel of non-animal proteins.

### SUMMARY

The present disclosure relates to the discovery that forming certain polyelectrolyte complexes of non-animal proteins can provide improved taste (namely, by masking off notes) and improved mouthfeel relative to the use of the non-animal protein without including it in such polyelectrolyte complexes.

In a first aspect, the disclosure provides polyelectrolyte complexes, comprising (a) a non-animal protein having a net positive charge (i.e., a polycationic non-animal protein), and (b) a polyionic polymer having a net negative charge (i.e., a polyanionic polymer). In some embodiments, the polycationic non-animal protein is a plant protein, such as pea protein, soy protein, canola protein, fava bean protein, and the like. In some embodiments, the polyanionic polymer is a pectin, gum Arabic, carrageenan, sunflower protein, and the like. In some embodiments, the polyelectrolyte complexes are in the form of a solid precipitate, which, for example, is formed by precipitating the complexed polymers out of an aqueous medium under acidic conditions. In some embodiments, the polyelectrolyte complexes are core-shell coacervates, where the shell portion comprises the polyelectrolyte complex.

In a second aspect, the disclosure provides uses of a polyanionic polymer to reduce one or more off notes of a polycationic non-animal protein. In some embodiments, the use comprises forming a polyelectrolyte complex between the polyanionic polymer and the polycationic non-animal protein. In some embodiments, the reduced off notes include a nutty note, a cereal note, a beany note, an astringent note, a bitter note, a sour note, a green vegetal note, an acidic note, or any combination thereof. In certain relates aspects, the disclosure provides methods of reducing one or more off notes of a polycationic non-animal protein, the methods comprising forming a polyelectrolyte complex between the polyanionic polymer and the polycationic non-animal protein. In some embodiments, the polycationic non-animal protein is a plant protein, such as pea protein, soy protein, fava bean protein, and the like. In some embodiments, the polyanionic polymer is a pectin, gum Arabic, sunflower protein, and the like. In some embodiments, the polyelectrolyte complexes are in the form of a solid precipitate, which, for example, is formed by precipitating the complexed polymers out of an aqueous medium under acidic conditions.

In a third aspect, the disclosure provides uses of a polyanionic polymer to enhance a mouthfeel of or enhance a texture of a polycationic non-animal protein. In some embodiments, enhancing a texture comprises improving the perceived thickness of an ingestible composition. In some embodiments, the use comprises forming a polyelectrolyte complex between the polyanionic polymer and the polycationic non-animal protein. In certain relates aspects, the disclosure provides methods of enhancing a mouthfeel of a polycationic non-animal protein, the methods comprising forming a polyelectrolyte complex between the polyanionic polymer and the polycationic non-animal protein. In some embodiments, the polycationic non-animal protein is a plant protein, such as pea protein, soy protein, fava bean protein, and the like. In some embodiments, the polyanionic polymer is a pectin, gum Arabic, sunflower protein, and the like. In some embodiments, the polyelectrolyte complexes are in the form of a solid precipitate, which, for example, is formed by precipitating the complexed polymers out of an aqueous medium under acidic conditions.

In a fourth aspect, the disclosure provides an ingestible composition, which comprises a polyelectrolyte complex of the first aspect. In some embodiments, the ingestible composition comprises a sweetener, a sweetness enhancer, an umami tastant, an umami enhancer, a kokumi tastant, a bitterness blocker, a flavoring, or any combination thereof. In some embodiments, the ingestible composition comprises a fiber component.

In a fifth aspect, the disclosure provides a flavored product comprising an ingestible composition of the fourth aspect. In some embodiments, the flavored product is a beverage product, such as a dairy or dairy analogue product. In some other embodiments, the flavored product is a food product, such as yogurt, or a meat analogue product, such as a chicken analogue product, a beef analogue product, or the like, or a seafood analogue product, such as a shellfish analogue product. In some embodiments, the flavored products are animal feed product, such as a cat feed or dog feed product.

Further aspects, and embodiments thereof, are set forth below in the Drawings, Detailed Description, the Abstract, and the Claims.

### BRIEF DESCRIPTION OF THE DRAWINGS

The following drawings are provided for purposes of illustrating various embodiments of the compositions and methods disclosed herein. The drawings are provided for illustrative purposes only and are not intended to describe any preferred compositions or preferred methods, or to serve as a source of any limitations on the scope of the claimed inventions.

FIG. 1 shows three micrographs with 20× magnification of the precipitate after 30 minutes of agitation, 1 hour of agitation, and 3 hours of agitation.

FIG. 2 shows three micrographs with 20× magnification of the precipitate after 30 minutes of agitation, 1 hour of agitation, and 3 hours of agitation.

FIG. 3 shows a micrograph with 20× magnification of the precipitate after complete formation.

FIG. 4 shows a micrograph with 20× magnification of the precipitate after formation.

FIG. 5 shows a micrograph with 40× magnification of the particles formed after spray drying.

FIG. 6 shows a micrograph with 40× magnification of the particles formed after spray drying.

FIG. 7 shows a micrograph with 40× magnification of the precipitate after formation.

FIG. 8 shows a micrograph with 40× magnification of the microparticle after formation and enzyme deactivation.

FIG. 9 shows micrographs of the microparticles formed after inactivation step at magnification 40×, with case a on the left and case b on the right.

FIG. 10 shows a micrograph is taken after inactivation, magnification 40×.

FIG. 11 shows micrographs at 40× magnification for the microparticles formed for each of the four cases after inactivation.

FIG. 12 shows micrographs of the formed complexes a) pea protein isolate/canola protein isolate; b) pea protein isolate/potato protein isolate; c) pea protein isolate/soluble wheat gluten).

FIG. 13 shows a micrograph of the formed particles, where the lower bar represents 50 microns.

FIG. 14 shows a micrograph of the formed emulsions, where the lower bar represents 50 microns.

### DETAILED DESCRIPTION

The following Detailed Description sets forth various aspects and embodiments provided herein. The description is to be read from the perspective of the person of ordinary skill in the relevant art. Therefore, information that is well known to such ordinarily skilled artisans is not necessarily included.

### Definitions

The following terms and phrases have the meanings indicated below, unless otherwise provided herein. This disclosure may employ other terms and phrases not expressly defined herein. Such other terms and phrases have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary

As used herein, “sweetener,” “sweet flavoring agent,” “sweet flavor entity,” or “sweet compound” all refer to a compound that elicits a detectable sweet flavor in a subject, such as a compound that activates a human T1R2 or T1R3 receptor in the course of in vitro screening or that is reported to be sweet via sensory evaluation by human subjects.

As used herein, “sweetener” or “sweet tastant” refers to a compound that elicits a detectable sweet flavor in a subject, such as a compound that activates a human T1R2 or T1R3 receptor in the course of in vitro screening or that is reported to be savory via sensory evaluation by human subjects.

As used herein, “umami tastant” refers to a compound that elicits a detectable umami flavor in a subject, such as a compound that activates a human T1R1 or T1R3 receptor in the course of in vitro screening or that is reported to be savory via sensory evaluation by human subjects.

As used herein, “bitter tastant” refers to a compound that elicits a detectable bitter flavor in a subject, such as a compound that activates one or more human T2R receptors in the course of in vitro screening or that is reported to be savory via sensory evaluation by human subjects.

As used herein, a “polyanionic polymer” is a polymer having multiple anionic functional groups within a polyelectrolyte complex. Such a polyanionic polymer has a lower isoelectric point that the polycationic polymer.

As used herein, a “polycationic polymer” is a polymer having multiple cationic functional groups within a polyelectrolyte complex. A “polycationic protein” refers to such a polymer where the polymer is a protein, such as a non-animal protein or a plant protein. Such a polycationic polymer has a higher isoelectric point that the polyanionic polymer.

As used herein, a “polyelectrolyte complex” refers to an association complex formed between a polyanionic polymer and a polycationic polymer. Such association complexes can be precipitated as particles, such as microparticles, or used to form part of the shell material for a core-shell microcapsule in the form of a coacervate.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure and are not meant to be limiting in any fashion. Nor do these phrases indicate any kind of preference for the disclosed embodiment.

As used herein, “comprise” or “comprises” or “comprising” or “comprised of” refer to groups that are open, meaning that the group can include additional members in addition to those expressly recited. For example, the phrase, “comprises A” means that A must be present, but that other members can be present too. The terms “include,” “have,” and “composed of” and their grammatical variants have the same meaning. In contrast, “consist of” or “consists of” or “consisting of” refer to groups that are closed. For example, the phrase “consists of A” means that A and only A is present.

As used herein, “optionally” means that the subsequently described event(s) may or may not occur. In some embodiments, the optional event does not occur. In some other embodiments, the optional event does occur one or more times.

As used herein, “or” is to be given its broadest reasonable interpretation and is not to be limited to an either/or construction. Thus, the phrase “comprising A or B” means that A can be present and not B, or that B is present and not A, or that A and B are both present. Further, if A, for example, defines a class that can have multiple members, e.g., A₁ and A₂, then one or more members of the class can be present concurrently.

Other terms are defined in other portions of this description, even though not included in this subsection.

### Polyelectrolyte Complexes

In certain aspects, the disclosure provides polyelectrolyte complexes comprising a polycationic non-animal protein and a polyanionic polymer. In these complexes, the polycationic non-animal protein and the polyanionic polymer associate with each other due to their opposite net charge.

Any suitable polycationic non-animal protein can be used, such as polycationic proteins derived from plants, algae, and fungi. In some embodiments, the polycationic non-animal protein is a polycationic plant protein. Non-limiting examples of such plant proteins include pea protein, soy protein, fava bean protein, and potato protein. In some embodiments, the polycationic non-animal protein is pea protein, soy protein, or a combination thereof. In some embodiments, the polycationic non-animal protein is pea protein. In some embodiments, the polycationic non-animal protein is soy protein. In some embodiments, the polycationic non-animal protein is fava bean protein. In some embodiments, the polycationic non-animal protein is potato protein, such as a potato protein comprising or consisting essentially of patatin.

In the polyelectrolyte complexes disclosed herein, the polycationic non-animal protein is associated with a polyanionic polymer. Any polyanionic protein suitable for comestible use is acceptable. Non-limiting examples of such polymers include pectin, gum Arabic, carrageenan, or certain plant proteins, such as sunflower protein and mung bean protein. In some embodiments, the polyanionic polymer is pectin. In some embodiments, the polyanionic polymer is gum Arabic. In some embodiments, the polyanionic polymer is carrageenan. In such embodiments, any suitable carrageenan can be used, such as iota-carrageenan, kappa-carrageenan, lambda-carrageenan, or any combinations thereof. In some embodiments, the polyanionic polymer is a plant protein. Note that plant proteins can be polyanionic or polycationic, depending on whether the pH is below its isoelectric point (polycationic) or above its isoelectric point (polyanionic). In some embodiments, the polyanionic polymer is sunflower protein. In some embodiments, the polyanionic polymer is mung bean protein. In some embodiments, the polyanionic polymer is pea protein. In some embodiments, the polyanionic polymer is mung bean protein, such as a potato protein comprising or consisting essentially of patatin.

As noted, most proteins can serve as either the polyanionic polymer or the polycationic non-animal protein, depending on whether it is disposed in an aqueous medium having a pH above or below its isoelectric point. Thus, depending on the pH at which the polyelectrolytes are formed, either the polyanionic polymer or the polycationic non-animal protein could be pea protein, soy protein, canola protein, hemp protein, potato protein, mung bean protein, sunflower protein, fava bean protein, wheat gluten, algae protein, and the like.

The polycationic non-animal protein and the polyanionic polymer can be present in the polyelectrolyte complex in any suitable weight ratio relative to each other. For example, in some embodiments, the weight ratio of polycationic non-animal protein to polyanionic polymer in the polyelectrolyte complex ranges from 1:10 to 10:1, or from 1:5 to 5:1. In some other embodiments, the weight ratio of polycationic non-animal protein to polyanionic polymer in the polyelectrolyte complex ranges from 1:1 to 10:1, or from 1:1 to 5:1.

The polyelectrolyte complexes may be formed in any suitable way. For example, in some embodiments, where the polyelectrolyte complexes are a solid, the polyelectrolyte complexes are a precipitate formed in an aqueous medium. This precipitation can be carried out at any suitable pH, but typically at a pH below the point of neutral charge (and lowest aqueous solubility) for both polymers. In some embodiments, the precipitate is formed at a pH ranging from 2.5 to 5.0, or from 2.5 to 4.5, or from 3.0 to 4.0. In such cases, the resulting precipitate is a particle. Such particles can be subjected to milling and other process to create a more uniform particle size and to reduce clumping and agglomeration.

These resulting particles typically have a zeta potential when present in an aqueous medium. In some embodiments, the particles of the polyelectrolyte complex have a zeta potential in water ranging from −30 mV to 30 mV, or from −20 mV to 20 mV, or from −10 mV to 10 mV.

In some embodiments, the particles comprise polyanionic and polycationic polymers that are cross-linked, for example, by introduction of an enzyme, such as a transglutaminase enzyme to cross-link the polymers.

Polycationic plant proteins have certain off notes, such as a beany note, a nutty note, a cereal note, an astringent note, a green vegetal note, a bitter note, a sour note, or an acidic note, that is perceived by many consumers as undesirable. It was surprisingly discovered that the polyelectrolyte complexes containing these plant proteins do not exhibit such off notes when consumed to the same degree as the uncomplexed plant protein. Thus, forming polyelectrolyte complexes, such as those described above, provides a suitable way of reducing the off notes of polycationic plant proteins. Thus, in certain aspects, the disclosure provides the use of polyanionic polymers (according to any of the embodiments set forth above) to reduce one or more off notes of a polycationic non-animal protein (according to any of the embodiments set forth above). In some embodiments, such use comprises forming a polyelectrolyte complex between the polycationic non-animal protein and the polyanionic polymer. In some related aspects, the disclosure provides methods of reducing one or more off notes of a polycationic non-animal protein (according to any of the embodiments set forth above), the method comprising forming a polyelectrolyte complex between the polycationic non-animal protein and the polyanionic polymer (according to any of the embodiments set forth above). These off notes include a nutty note, a cereal note, and a beany note, an astringent note, a bitter note, a green vegetal note, a sour note, an acidic note, as well as certain vegetal and green notes. Such notes include both aroma and taste.

Polycationic plant proteins also tend to have a mouthfeel that is undesirable to many consumers. For example, many consumers perceive such proteins as having a certain chalky texture that tends to cause products containing such proteins to be perceived as dry or insufficiently juicy. It was surprisingly discovered that the polyelectrolyte complexes containing these plant proteins exhibit improved mouthfeel in comparison to the uncomplexed plant protein. In some cases, this is perceived as an enhanced juiciness. Thus, forming polyelectrolyte complexes, such as those described above, provides a suitable way of reducing the off notes of polycationic plant proteins. Thus, in certain aspects, the disclosure provides the use of polyanionic polymers (according to any of the embodiments set forth above) to enhance mouthfeel or juiciness of a polycationic non-animal protein (according to any of the embodiments set forth above). In some embodiments, such use comprises forming a polyelectrolyte complex between the polycationic non-animal protein and the polyanionic polymer. In some related aspects, the disclosure provides methods of enhancing mouthfeel or juiciness or perceived thickness of a polycationic non-animal protein (according to any of the embodiments set forth above), the method comprising forming a polyelectrolyte complex between the polycationic non-animal protein and the polyanionic polymer (according to any of the embodiments set forth above).

When the polyelectrolyte complexes are incorporated into ingestible compositions, they can be used in similar concentrations as the uncomplexed protein would be used in such compositions. In some other cases, a lower amount can be used, such as in instances where only a portion of the uncomplexed protein is replaced with the polyelectrolyte complex or in instances where the polyanionic polymer is also a protein.

### Methods of Microparticle Formation

The microparticles comprising the polyelectrolyte complexes can be formed in any suitable way. In some embodiments, a first polymer and a second polymer are introduced into an aqueous medium, wherein the first polymer has a first isoelectric point and the second polymer has a second isoelectric point, which is higher than the first isoelectric point. In some such embodiments, the pH of the aqueous medium is adjusted to a pH between the first isoelectric point and the second isoelectric point, thereby causing the first polymer to become a polyanionic polymer and the second polymer to become a polycationic polymer. In some embodiments, the polyanionic polymer and the polycationic polymer are allowed to associate in the aqueous medium, and precipitate to form microparticles. Such particles can be subjected to milling and other process to create a more uniform particle size and to reduce clumping and agglomeration. In some other embodiments, the association of the polyanionic polymer and the polycationic polymer occurs in the presence of an enzyme, such as a transglutaminase enzyme, and, optionally, an oil, such as a fatty acid glyceride. Suitable fatty acid glycerides include glycerides of capric acid, caprylic acid, or any combinations thereof.

### Ingestible Compositions

In certain aspects, the disclosure provides ingestible compositions comprising a polyelectrolyte complex according to any of the embodiments set forth in the previous section. The polyelectrolyte complex can be present in any suitable concentration within the ingestible composition. For example, in some embodiments, the polyelectrolyte complex makes up from 10% by weight to 99% by weight, or from 20% by weight to 95% by weight, or from 30% by weight to 95% by weight, of the ingestible composition, based on the total dry weight of the ingestible composition.

The ingestible composition can include other ingredients. These additional ingredients can be part of the ingestible composition.

### Other Non-Animal Proteins

In certain embodiments, the ingestible compositions comprise one or more other non-animal proteins that are not in complexed form. These other non-animal proteins include, without limitation, plant proteins, algal proteins, mycoproteins, or combinations thereof. In some embodiments, the other non-animal proteins are plant-based protein. Non-limiting examples of such plant proteins include hemp protein, almond protein, cashew protein, canola (rapeseed) protein, chickpea protein, wheat protein, potato protein, lupine, rice protein, pea protein, soy protein, fava bean protein, mung bean protein, sunflower protein, red lentil protein, oat protein, or any combination thereof. These other non-animal proteins, when present, can make up any suitable proportion of the ingestible composition. For example, in some embodiments, the other non-animal protein makes up from 1 percent by weight to 50 percent by weight, or from 1 percent by weight to 40 percent by weight, or from 1 percent by weight to 30 percent by weight, or from 1 percent by weight to 20 percent by weight, based on the total dry weight of the ingestible composition.

### Fibers

In some embodiments, the ingestible composition includes one or more fibers. Such fibers are generally plant-derived and include both soluble and insoluble fibers.

As used herein, the term “soluble fiber” refers to polysaccharides characterized as being soluble by using the method of the Association of Official Analytical Chemists (AOAC) and as set forth in Prosky et al., J. ASSOC. OFF. ANAL. CHEM., vol. 70(5), pp. 1017-1023 (1988). Any suitable soluble fibers can be used, including, but not limited to, fruit fiber (such as citrus fiber), grain fibers, psyllium husk fiber, natural soluble fibers and synthetic soluble fibers. Natural fibers include soluble corn fiber, maltodextrin, acacia, and hydrolyzed guar gum. Synthetic soluble fibers include polydextrose, modified food starch, and the like. Non-limiting examples of food-grade sources of soluble fiber include inulin, corn fiber, barley fiber, corn germ, ground oat hulls, milled corn bran, derivatives of the aleurone layer of wheat bran, flax flour, whole flaxseed bran, winter barley flake, ground course kilned oat groats, maize, pea fiber (e.g. Canadian yellow pea), Danish potatoes, konjac vegetable fiber (glucomannan), psyllium fiber from seed husks of planago ovate, psyllium husk, liquid agave fiber, rice bran, oat sprout fibers, amaranth sprout, lentil flour, grape seed fiber, apple, blueberry, cranberry, fig fibers, ciranda power, carob powder, milled prune fiber, mango fiber, apple fiber, orange, orange pulp, strawberry, carrageenan hydrocolloid, derivatives of eucheuma cottonnil seaweed, cottonseed, soya, kiwi, acacia gum fiber, bamboo, chia, potato, potato starch, pectin (carbohydrate) fiber, hydrolyzed guar gum, carrot, soy, soybean, chicory root, oat, wheat, tomato, polydextrose fiber, refined corn starch syrup, isomalto-oligosaccharide mixtures, soluble dextrin, mixtures of citrus bioflavonoids, cell-wall broken nutritional yeast, lipophilic fibers, plum juice, derivatives from larch trees, olygose fibers, derivatives from cane sugar, short-chain fructooligosaccharides, synthetic polymers of glucose, polydextrose, pectin, polanion compounds, cellulose fibers, cellulose fibers derived from hard wood plants and carboxymethyl cellulose.

In some embodiments, the ingestible composition or the protein additive composition can also include certain insoluble fibers, which can provide structure and texture to the ingestible composition. Any suitable insoluble fiber can be used. In some embodiments, the insoluble fiber is a plant-derived fiber. Non-limiting examples include nut fibers, grain fibers, rice fibers, seed fibers, oat fibers, pea fibers, potato fibers, berry fibers, soybean fibers, banana fibers, citrus fibers, apple fibers, and carrot fibers. In some embodiments, the insoluble fiber is pea fiber.

In some embodiments, the ingestible composition comprises pea fiber, citrus fiber, potato fiber, psyllium fiber, acacia fiber, inulin, konjac fiber, or any combination thereof.

The fiber can make up any suitable proportion of the ingestible composition. For example, in some embodiments, the fiber makes up from 1% by weight to 50% by weight, or from 1% by weight to 40% by weight, or from 1% by weight to 30% by weight, or from 1% by weight to 20% by weight, or from 3% by weight to 50% by weight, or from 3% by weight to 40% by weight, or from 3% by weight to 30% by weight, or from 3% by weight to 20% by weight, based on the total dry weight of the ingestible composition.

### Flavorings, Extracts, and Flavor and Aroma Modifiers

In some embodiments, the ingestible composition includes one or more flavorings, extracts, flavor modifiers, aroma modifiers, or any combination thereof.

In some embodiments, the ingestible compositions disclosed herein comprise a flavoring. In general, the flavoring improves the taste and flavor of the ingestible composition or the resulting flavored product in which the ingestible composition is used. Such improvement includes reducing the bitterness of the ingestible composition or the resulting flavored product, reducing the perception of astringency of the ingestible composition or the resulting flavored product, reducing the perception of green taste notes (such as pea taste) of the ingestible composition or the resulting flavored product, reducing the perception of cereal notes of the ingestible composition or the resulting flavored product, improving the perception of creaminess of the ingestible composition or the resulting flavored product, improving the perception of fattiness of the ingestible composition or the resulting flavored product, improving the perception of sweetness of the ingestible composition or the resulting flavored product, improving the perception of savory taste (umami or kokumi) of the ingestible composition or the resulting flavored product, improving the mouthfeel or mouthcoating or perceived thickness of the ingestible composition or the resulting flavored product, improving the perception of juiciness of the ingestible composition or the resulting flavored product, improving the perception of thickness of the ingestible composition or the resulting flavored product.

Any suitable flavoring can be used. In some embodiments, the flavoring comprises synthetic flavor oils and flavoring aromatics or oils, oleoresins and extracts derived from plants, leaves, flowers, fruits, and so forth, or combinations thereof. Non-limiting examples of flavor oils include spearmint oil, cinnamon oil, oil of wintergreen (methyl salicylate), peppermint oil, Japanese mint oil, clove oil, bay oil, anise oil, eucalyptus oil, thyme oil, cedar leaf oil, oil of nutmeg, allspice, oil of sage, mace, oil of bitter almonds, and cassia oil. Non-limiting examples of other flavors include natural and synthetic fruit flavors such as vanilla, and citrus oils including lemon, orange, lime, grapefruit, yazu, sudachi, and fruit essences including apple, pear, peach, grape, blueberry, strawberry, raspberry, cherry, plum, pineapple, watermelon, apricot, banana, melon, apricot, ume, cherry, raspberry, blackberry, tropical fruit, mango, mangosteen, pomegranate, papaya and so forth. Other potential flavors include a milk flavor, a butter flavor, a cheese flavor, a cream flavor, and a yogurt flavor; a vanilla flavor; tea or coffee flavors, such as a green tea flavor, a oolong tea flavor, a tea flavor, a cocoa flavor, a chocolate flavor, and a coffee flavor; mint flavors, such as a peppermint flavor, a spearmint flavor, and a Japanese mint flavor; spicy flavors, such as an asafetida flavor, an ajowan flavor, an anise flavor, an angelica flavor, a fennel flavor, an allspice flavor, a cinnamon flavor, a chamomile flavor, a mustard flavor, a cardamom flavor, a caraway flavor, a cumin flavor, a clove flavor, a pepper flavor, a coriander flavor, a sassafras flavor, a savory flavor, a Zanthoxyli Fructus flavor, a perilla flavor, a juniper berry flavor, a ginger flavor, a star anise flavor, a horseradish flavor, a thyme flavor, a tarragon flavor, a dill flavor, a capsicum flavor, a nutmeg flavor, a basil flavor, a marjoram flavor, a rosemary flavor, a bayleaf flavor, and a wasabi (Japanese horseradish) flavor; alcoholic flavors, such as a wine flavor, a whisky flavor, a brandy flavor, a rum flavor, a gin flavor, and a liqueur flavor; floral flavors; and vegetable flavors, such as an onion flavor, a garlic flavor, a cabbage flavor, a carrot flavor, a celery flavor, mushroom flavor, and a tomato flavor. These flavoring agents may be used in liquid or solid form and may be used individually or in admixture. In the context of dairy or dairy analog products, the most commonly used flavor agents are agents that impart flavors such as vanilla, French vanilla, chocolate, banana, lemon, hazelnut, coconut, almond, strawberry, mocha, coffee, tea, chai, cinnamon, caramel, cream, brown sugar, toffee, pecan, butter pecan, toffee, Irish creme, white chocolate, raspberry, pumpkin pie spice, peppermint, or any combination thereof.

In some embodiments, the flavoring is a flavoring that provides a meat or savory tonality, including flavorings or tonalities of beef, lamb, bison, smoke, pork, bacon, ham, sausage, chicken, turkey, goose, duck, mushroom, celery, tomato, onion, garlic, carrot, leek, fish, shellfish, soy, miso, and the like. In some further embodiments, the flavoring comprises one or more lactones, which impart a creamy flavor to the ingestible composition.

In some embodiments, the flavoring comprises a yeast extract, such as a yeast lysate. Such extracts can be obtained from any suitable yeast strain, where such extracts are suitable for human consumption. Non-limiting examples of such yeasts include: yeasts of the genus Saccharomyces, such as Saccharomyces cerevisiae or Saccharomyces pastorianus; yeasts of the genus Candida, such as Candida utilis; yeasts of the genus Kluyveromyces, such as Kluyveromyces lactis or Kluyveromyces marxianus; yeasts of the genus Pichia such as Pichia pastoris; yeasts of the genus Debaryomyces such as Debaryomyces hansenii; and yeasts of the genus Zygosaccharomyces such as Zygosaccharomyces mellis. In some embodiments, the yeast is a yeast collected after brewing beer, sake, or the like. In some embodiments, the yeast is a yeast subjected to drying treatment (dried yeast) after collection.

Such extracts can be produced by any suitable means. In general, yeast extracts or lysates are made by extracting the contents of the yeast cells from the cell wall material. In many instances, the digestive enzymes in the cells (or additional enzymes added to the composition) break down the proteins and polynucleotides in the yeast to amino acids, oligopeptides (for example, from 2 to 10 peptides), nucleotides, oligonucleotides (from 2 to 10 nucleotides), and mixtures thereof. A yeast lysate can be prepared by lysing a yeast. For example, in some embodiments, the yeast after culture is crushed or lysed by an enzymatic decomposition method, a self-digestion method, an alkaline extraction method, a hot water extraction method, an acid decomposition method, an ultrasonic crushing method, crushing with a homogenizer, a freezing-thawing method, or the like (two or more thereof may be used in combination), whereby a yeast lysate is obtained. Yeast may be cultured by a conventional method. In some embodiments, the yeast after culture is heat-treated and then treated with a lytic enzyme to obtain an enzyme lysate. The conditions for the heat treatment are, for example, 80° C. to 90° C. for 5 minutes to 30 minutes. As the lytic enzyme used for the enzymatic decomposition method, various enzymes can be used as long as they can lyse the cell wall of yeast. The reaction conditions may be set so as to be optimum or suitable for the lytic enzyme(s) to be used, and specific examples thereof can include a temperature of 50° C. to 60° C., and a pH of 7.0 to 8.0. The reaction time is also not particularly limited, and can be, for example, 3 hours to 5 hours.

Compositions comprising yeast lysate can be obtained from a variety of commercial sources. For example, in some embodiments, the yeast lysate is provides by the flavoring additive sold under the name MODUMAX (DSM Food Specialties BV, Delft, Netherlands).

The flavoring also includes, in certain embodiments, one or more additional flavor-modifying compounds, such as compounds that enhance sweetness (e.g., phloretin, naringenin, glucosylated steviol glycosides, etc.), compounds that block bitterness, compounds that enhance umami, compounds that enhance kokumi, compounds that reduce sourness or licorice taste, compounds that enhance saltiness, compounds that enhance a cooling effect, compounds that enhance mouthfeel, or any combinations of the foregoing.

In some embodiments, the ingestible composition comprises a sweetener. The sweetener can be present in any suitable concentration, depending on factors such as the sweetener's potency as a sweetener, its solubility, and the like.

In general, the ingestible compositions disclosed herein can include any suitable sweeteners or combination of sweeteners. In some embodiments, the sweetener is a common saccharide sweeteners, such as sucrose, fructose, glucose, and sweetener compositions comprising natural sugars, such as corn syrup (including high fructose corn syrup) or other syrups or sweetener concentrates derived from natural fruit and vegetable sources. In some embodiments, the sweetener is sucrose, fructose, or a combination thereof. In some embodiments, the sweetener is sucrose. In some other embodiments, the sweetener is selected from rare natural sugars including D-allose, D-psicose, L-ribose, D-tagatose, L-glucose, L-fucose, L-arbinose, D-turanose, and D-leucrose. In some embodiments, the sweetener is selected from semi-synthetic “sugar alcohol” sweeteners such as erythritol, isomalt, lactitol, mannitol, sorbitol, xylitol, maltodextrin, and the like. In some embodiments, the sweetener is selected from artificial sweeteners such as aspartame, saccharin, acesulfame-K, cyclamate, sucralose, and alitame. In some embodiments, the sweetener is selected from the group consisting of cyclamic acid, mogroside, tagatose, maltose, galactose, mannose, sucrose, fructose, lactose, allulose, neotame and other aspartame derivatives, glucose, D-tryptophan, glycine, maltitol, lactitol, isomalt, hydrogenated glucose syrup (HGS), hydrogenated starch hydrolyzate (HSH), stevioside, rebaudioside A, other sweet Stevia-based glycosides, chemically modified steviol glycosides (such as glucosylated steviol glycosides), mogrosides, chemically modified mogrosides (such as glucosylated mogrosides), carrelame and other guanidine-based sweeteners. In some embodiments, the additional sweetener is a combination of two or more of the sweeteners set forth in this paragraph. In some embodiments, the sweetener may combinations of two, three, four or five sweeteners as disclosed herein. In some embodiments, the additional sweetener is a sugar. In some embodiments, the additional sweetener is a combination of one or more sugars and other natural and artificial sweeteners. In some embodiments, the additional sweetener is a sugar. In some embodiments, the sugar is cane sugar. In some embodiments, the sugar is beet sugar. In some embodiments, the sugar may be sucrose, fructose, glucose or combinations thereof. In some embodiments, the sugar is sucrose. In some embodiments, the sugar is a combination of fructose and glucose.

In some embodiments, the sweeteners can also include, for example, sweetener compositions comprising one or more natural or synthetic carbohydrate, such as corn syrup, high fructose corn syrup, high maltose corn syrup, glucose syrup, sucralose syrup, hydrogenated glucose syrup (HGS), hydrogenated starch hydrolyzate (HSH), or other syrups or sweetener concentrates derived from natural fruit and vegetable sources, or semi-synthetic “sugar alcohol” sweeteners such as polyols. Non-limiting examples of polyols in some embodiments include erythritol, maltitol, mannitol, sorbitol, lactitol, xylitol, isomalt, propylene glycol, glycerol (glycerin), threitol, galactitol, palatinose, reduced isomalto-oligosaccharides, reduced xylo-oligosaccharides, reduced gentio-oligosaccharides, reduced maltose syrup, reduced glucose syrup, isomaltulose, maltodextrin, and the like, and sugar alcohols or any other carbohydrates or combinations thereof capable of being reduced which do not adversely affect taste.

The sweetener may be a natural or synthetic sweetener that includes, but is not limited to, agave inulin, agave nectar, agave syrup, amazake, brazzein, brown rice syrup, coconut crystals, coconut sugars, coconut syrup, date sugar, fructans (also referred to as inulin fiber, fructo-oligosaccharides, or oligo-fructose), green stevia powder, Stevia rebaudiana, rebaudioside A, rebaudioside B, rebaudioside C, rebaudioside D, rebaudioside E, rebaudioside F, rebaudioside I, rebaudioside H, rebaudioside L, rebaudioside K, rebaudioside J, rebaudioside N, rebaudioside O, rebaudioside M and other sweet stevia-based glycosides, stevioside, stevioside extracts, honey, Jerusalem artichoke syrup, licorice root, luo han guo (fruit, powder, or extracts), lucuma (fruit, powder, or extracts), maple sap (including, for example, sap extracted from Acer saccharum, Acer nigrum, Acer rubrum, Acer saccharinum, Acer platanoides, Acer negundo, Acer macrophyllum, Acer grandidentatum, Acer glabrum, Acer mono), maple syrup, maple sugar, walnut sap (including, for example, sap extracted from Juglans cinerea, Juglans nigra, Juglans ailatifolia, Juglans regia), birch sap (including, for example, sap extracted from Betula papyrifera, Betula alleghaniensis, Betula lenta, Betula nigra, Betula populifolia, Betula pendula), sycamore sap (such as, for example, sap extracted from Platanus occidentalis), ironwood sap (such as, for example, sap extracted from Ostrya virginiana), mascobado, molasses (such as, for example, blackstrap molasses), molasses sugar, monatin, monellin, cane sugar (also referred to as natural sugar, unrefined cane sugar, or sucrose), palm sugar, panocha, piloncillo, rapadura, raw sugar, rice syrup, sorghum, sorghum syrup, cassava syrup (also referred to as tapioca syrup), thaumatin, yacon root, malt syrup, barley malt syrup, barley malt powder, beet sugar, cane sugar, crystalline juice crystals, caramel, carbitol, carob syrup, castor sugar, hydrogenated starch hydrolates, hydrolyzed can juice, hydrolyzed starch, invert sugar, anethole, arabinogalactan, arrope, syrup, P-4000, acesulfame potassium (also referred to as acesulfame K or ace-K), alitame (also referred to as aclame), advantame, aspartame, baiyunoside, neotame, benzamide derivatives, bernadame, canderel, carrelame and other guanidine-based sweeteners, vegetable fiber, corn sugar, coupling sugars, curculin, cyclamates, cyclocarioside I, demerara, dextran, dextrin, diastatic malt, dulcin, sucrol, valzin, dulcoside A, dulcoside B, emulin, enoxolone, maltodextrin, saccharin, estragole, ethyl maltol, glucin, gluconic acid, glucono-lactone, glucosamine, glucoronic acid, glycerol, glycine, glycyphillin, glycyrrhizin, glycyrrhetic acid monoglucuronide, golden sugar, yellow sugar, golden syrup, granulated sugar, gynostemma, hernandulcin, isomerized liquid sugars, jallab, chicory root dietary fiber, kynurenine derivatives (including N′-formyl-kynurenine, N′-acetyl-kynurenine, 6-chloro-kynurenine), galactitol, litesse, ligicane, lycasin, lugduname, guanidine, falernum, mabinlin I, mabinlin II, maltol, maltisorb, maltodextrin, maltotriol, mannosamine, miraculin, mizuame, mogrosides (including, for example, mogroside IV, mogroside V, and neomogroside), mukurozioside, nano sugar, naringin dihydrochalcone, neohesperidine dihydrochalcone, nib sugar, nigero-oligosaccharide, norbu, orgeat syrup, osladin, pekmez, pentadin, periandrin I, perillaldehyde, perillartine, petphyllum, phenylalanine, phlomisoside I, phlorodizin, phyllodulcin, polyglycitol syrups, polypodoside A, pterocaryoside A, pterocaryoside B, rebiana, refiners syrup, rub syrup, rubusoside, selligueain A, shugr, siamenoside I, siraitia grosvenorii, soybean oligosaccharide, Splenda, SRI oxime V, steviol glycoside, steviolbioside, stevioside, strogins 1, 2, and 4, sucronic acid, sucrononate, sugar, suosan, phloridzin, superaspartame, tetrasaccharide, threitol, treacle, trilobtain, tryptophan and derivatives (6-trifluoromethyl-tryptophan, 6-chloro-D-tryptophan), vanilla sugar, volemitol, birch syrup, aspartame-acesulfame, assugrin, and combinations or blends of any two or more thereof.

In still other embodiments, the sweetener can be a chemically or enzymatically modified natural high potency sweetener. Modified natural high potency sweeteners include glycosylated natural high potency sweetener such as glucosyl-, galactosyl-, or fructosyl-derivatives containing 1-50 glycosidic residues. Glycosylated natural high potency sweeteners may be prepared by enzymatic transglycosylation reaction catalyzed by various enzymes possessing transglycosylating activity. In some embodiments, the modified sweetener can be substituted or unsubstituted.

In some embodiments, the flavoring comprises one or more sweetness enhancing compounds. Such sweetness enhancing compounds include, but are not limited to, naturally derived compounds, such as hesperitin dihydrochalcone, hesperitin dihydrochalcone-4-O′glucoside, neohesperitin dihydrochalcone, brazzein, hesperidin, phyllodulcin, naringenin, naringin, phloretin, glucosylated steviol glycosides, (2R,3R)-3-acetoxy-5,7,4′-trihydroxyflavanone, (2R,3R)-3-acetoxy-5,7,3′-trihydroxy-4′-methoxyflavanone, rubusosides, eriodictyol, homoeriodictyol, or synthetic compounds, such as any compounds set forth in U.S. Pat. Nos. 8,541,421; 8,815,956; 9,834,544; 8,592,592; 8,877,922; 9,000,054; and 9,000,051, as well as U.S. Patent Application Publication No. 2017/0119032. As used herein, the term “glucosylated steviol glycoside” refers to the product of enzymatically glucosylating natural steviol glycoside compounds. The glucosylation generally occurs through a glycosidic bond, such as an α-1,2 bond, an α-1,4 bond, an α-1,6 bond, a β-1,2 bond, a β-1,4 bond, a β-1,6 bond, and so forth. In some embodiments of any of the preceding embodiments, the comestible composition comprises 3-((4-amino-2,2-dioxo-1H-benzo[c][1,2,6]thiadiazin-5-yl)oxy)-2,2-dimethyl-N-propyl-propanamide or N-(1-((4-amino-2,2-dioxo-1H-benzo[c][1,2,6]thiadiazin-5-yl)oxy)-2-methyl-propan-2-yl)isonicotinamide.

In some further embodiments, the flavoring comprises one or more umami enhancing compounds. Such umami enhancing compounds include, but are not limited to, naturally derived compounds, or synthetic compounds, such as any compounds set forth in U.S. Pat. Nos. 8,735,081; 8,124,121; and 8,968,708. In some embodiments, the umami-enhancing compound is (2R,4R)-1,2,4-trihydroxy-heptadec-16-ene, (2R,4R)-1,2,4-trihydroxyheptadec-16-yne, or a mixture thereof. In some embodiments, the umami-enhancing compound is (3R,5S)-1-(4-hydroxy-3-methoxyphenyl)decane-3,5-diol diacetate. In some embodiments, the umami-enhancing compound is N-(heptan-4-yl)benzo[d][1,3]dioxole-5-carboxamide.

In some embodiments, the ingestible composition comprises one or more compounds commonly used in savory products. Such flavorings include glutamates (such as MSG), arginates, avocadene, avocadyne, a purine ribonucleitide (such as inosine monophosphate (IMP), guanosine monophosphate (GMP), hypoxanthine, inosine), a yeast extract (as noted above), a fermented food product, cheese, garlic or extracts thereof, a gamma-glutamyl-containing polypeptide, a gamma-glutamyl-containing oligopeptide (such as gamma-glutamyl-containing tripeptides); an flavor-modifying composition (such as a cinnamic acid amide or a derivative thereof), a nucleotide, an oligonucleotide, a plant extract, a food extract, or any combinations thereof.

In some further embodiments, the flavoring comprises one or more cooling enhancing compounds. Such cooling enhancing compounds include, but are not limited to, naturally derived compounds, such as menthol or analogs thereof, or synthetic compounds, such as any compounds set forth in U.S. Pat. Nos. 9,394,287 and 10,421,727.

In some further embodiments, the flavoring comprises one or more bitterness blocking compounds. Such bitterness blocking compounds include, but are not limited to, naturally derived compounds, such as menthol or analogs thereof, or synthetic compounds, such as any compounds set forth in U.S. Pat. Nos. 8,076,491; 8,445,692; and 9,247,759, or in PCT Publication No. WO 2020/033669. In some embodiments, the bitterness blocking compound is 3-(1-((3,5-dimethylisoxazol-4-yl)-methyl)-1H-pyrazol-4-yl)-1-(3-hydroxybenzyl)-imidazolidine-2,4-dione.

In some further embodiments, the flavoring comprises one or more sour taste modulating compounds.

In some further embodiments, the flavoring comprises one or more mouthfeel modifying or mouthfeel enhancing compounds. Such mouthfeel modifying compounds include, but are not limited to, polymethoxylated flavones, tannins, cellulosic materials, bamboo powder, and the like.

In some further embodiments, the flavoring comprises one or more flavor masking compounds. Such flavor masking compounds include, but are not limited to, cellulosic materials, materials extracted from fungus, materials extracted from plants, citric acid, carbonic acid (or carbonates), and the like.

In some embodiments, the flavor-modifying compounds described above are included to improve other tastants that may be present in the comestible composition itself, or that may be included within the flavored products that employ such compositions. Such tastants include sweeteners, umami tastants, kokumi tastants, bitter tastants, sour tastants, and the like.

In some embodiments, the ingestible comprises one or more metal salts or metal complexes, such as iron salts or iron complexes. Such compounds can include any comestible metal salt or complex, such as salts or complexes of calcium, magnesium, sodium, potassium, iron, cobalt, copper, zinc, manganese, molybdenum, and selenium. In some embodiments, the iron compound is an iron salt or an iron complex. In some particular embodiments, the metal compound is a ferrous (Fe²⁺) salt or a ferrous (Fe²⁺) complex. In some embodiments, the metal compound is a ferrous (Fe²⁺) salt, such as ferrous sulfate, ferrous lactate, ferrous fumarate, ferrous gluconate, ferrous succinate, ferrous chloride, ferrous oxalate, ferrous nitrate, ferrous citrate, ferrous ascorbate, ferric citrate, ferric phosphate, or any combination thereof. In some other embodiments, the metal compound is a ferric (Fe³⁺) salt or a ferric (Fe³⁺) complex, such as ferric pyrophosphate. In some embodiments, the iron compound is ferrous lactate, ferrous sulfate, or any combination thereof.

In some embodiments, the iron compound is a heme-containing protein. As used herein, the term “heme containing protein” includes any polypeptide covalently or noncovalently bound to a heme moiety. In some embodiments, the heme-containing polypeptide is a globin and can include a globin fold, which comprises a series of seven to nine alpha helices. Globin type proteins can be of any class (for example, class I, class II, or class III), and in some embodiments, can transport or store oxygen. For example, a heme-containing protein can be a non-symbiotic type of hemoglobin or a leghemoglobin. A heme-containing polypeptide can be a monomer, such as a single polypeptide chain, or can be a dimer, a trimer, tetramer, and/or higher order oligomer. The lifetime of the oxygenated Fe²⁺ state of a heme-containing protein can be similar to that of myoglobin or can exceed it by 10%, or 20%, or 30%>, or 40%, or 50%, or even 100%, or more under conditions in which the heme-protein-containing consumable is manufactured, stored, handled or prepared for consumption.

Non-limiting examples of heme-containing proteins include an androglobin, a cytoglobin, a globin E, a globin X, a globin Y, a hemoglobin, a myoglobin, an erythrocruorin, a beta hemoglobin, an alpha hemoglobin, a protoglobin, a cyanoglobin, a cytoglobin, a histoglobin, a neuroglobins, a chlorocruorin, a truncated hemoglobin (e.g., HbN or HbO), a truncated 2/2 globin, a hemoglobin 3 (e.g., Glb3), a cytochrome, or a peroxidase.

Heme-containing proteins that can be used in the comestible compositions described herein and can be from mammals (for example, farm animals such as cows, goats, sheep, pigs, ox, or rabbits), birds, plants, algae, fungi (for example, yeast or filamentous fungi), ciliates, or bacteria. For example, a heme-containing protein can be from a mammal such as a farm animal (e.g., a cow, goat, sheep, pig, fish, ox, or rabbit) or a bird such as a turkey or chicken. Heme-containing proteins can be from a plant such as Nicotiana tabacum or Nicotiana sylvestris (tobacco); Zea mays (corn), Arabidopsis thaliana, a legume such as Glycine max (soybean), Cicer arietinum (garbanzo or chick pea), Pisum sativum (pea) varieties such as garden peas or sugar snap peas, Phaseolus vulgaris varieties of common beans such as green beans, black beans, navy beans, northern beans, or pinto beans, Vigna unguiculata varieties (cow peas), Vigna radiata (mung beans), Lupinus albus (lupin), or Medicago sativa (alfalfa); Brassica napus (canola), Triticum sps. (wheat, including wheat berries, and spelt); Gossypium hirsutum (cotton); Oryza sativa (rice); Zizania sps. (wild rice); Helianthus annuus (sunflower); Beta vulgaris (sugarbeet); Pennisetum glaucum (pearl millet); Chenopodium sp. (quinoa); Sesamum sp. (sesame); Linum usitatissimum (flax); or Hordeum vulgare (barley). Heme-containing proteins can be isolated from fungi such as Saccharomyces cerevisiae, Pichia pastoris, Magnaporthe oryzae, Fusarium graminearum, Aspergillus oryzae, Trichoderma reesei, Myceliopthera thermophile, Kluyveramyces lactis, or Fusarium oxysporum. Heme-containing proteins can be isolated from bacteria such as Escherichia coli, Bacillus subtilis, Bacillus licheniformis, Bacillus megaterium, Synechocistis sp., Aquifex aeolicus, Methylacidiphilum infernorum, or thermophilic bacteria such as Thermophilus spp. The sequences and structure of numerous heme-containing proteins are known. See, for example, Reedy, et al, Nucleic Acids Research, 2008, Vol. 36, Database issue D307-D313 and the Heme Protein Database available on the world wide web at http://hemeprotein.info/heme.php.

In some embodiments, a non-symbiotic hemoglobin can be from any plant. In some embodiments, a non-symbiotic hemoglobin can be from a plant selected from the group consisting of soybean, sprouted soybean, alfalfa, golden flax, black bean, black eyed pea, northern bean, tobacco, pea, garbanzo, moong bean, cowpeas, pinto beans, pod peas, quinoa, sesame, sunflower, wheat berries, spelt, barley, wild rice, and rice.

In some embodiments, the heme-containing protein is a leghemoglobin, such as a soy, pea, or cowpea leghemoglobin.

Heme-containing or other proteins also can be recombinantly produced using polypeptide expression techniques (e.g., heterologous expression techniques using bacterial cells, insect cells, fungal cells such as yeast, plant cells such as tobacco, soybean, or Arabidopsis, or mammalian cells). For example, leghemoglobin can be recombinantly produced in E. coli or Pichia pastoris. In some cases, standard polypeptide synthesis techniques (such as liquid-phase polypeptide synthesis techniques or solid-phase polypeptide synthesis techniques) can be used to produce heme-containing proteins synthetically. In some cases, in vitro transcription-translation techniques can be used to produce heme-containing proteins.

The heme-containing proteins or iron salts can be used at any suitable concentration. Examples are set forth in PCT Publication No. WO 2015/153666, which is incorporated herein by reference.

The iron compound can make up any suitable weight of the ingestible particle. In some embodiments, the iron compound makes up from 0.1 percent by weight to 10 percent by weight, or from 0.2 percent by weight to 5 percent by weight, or from 0.5 percent by weight to 3 percent by weight, of the ingestible composition, based on the total dry weight of the ingestible composition.

### Other Additives

In some embodiments, the ingestible composition comprises various other additives, such as emulsifiers, bulking agents, thickeners, and the like.

For example, in some embodiments, the ingestible composition comprises an emulsifier. Any suitable emulsifier can be used. For example, in some non-limiting embodiments, the emulsifier comprises lecithin, monoglycerides, diglycerides, polysorbates, vegetable oils, and the like. In some embodiments, the emulsifier comprises lecithin. Other examples of emulsifiers can be found in McCutcheon's Emulsifiers & Detergents or the Industrial Surfactants Handbook. The emulsifier can be present in any suitable concentration, which can be adjusted so as to form a stable emulsion of the other components in the comestible composition, for example, when incorporated into a flavored product.

In some instances, it may be desirable to include additives that assist in adjusting the viscosity of the ingestible composition (for example, when the ingestible composition is introduced into water or includes water). Various salts and acids can be used to carry out such adjustments. In some embodiments, the comestible composition or the resulting flavored product comprises one or more salts. Non-limiting examples of suitable salts include magnesium sulfate, sodium chloride, sodium sulfate, calcium chloride, calcium sulfate, potassium sulfate, potassium chloride, potassium sorbate, potassium phosphate, potassium monophosphate, zinc chloride, zinc sulfate, or any mixtures thereof. In some embodiments, the comestible composition or the resulting flavored product also comprises one or more acids, which may be used alone or in combination with the aforementioned salts. Non-limiting examples of suitable acids include citric acid, lactic acid, acetic acid, tartaric acid, succinic acid, ascorbic acid, maleic acid, phosphoric acid, monopotassium phosphate, gluconic acid, glucono-lactone, glucoronic acid, glycyrrhetic acid, folic acid, pantothenic acid or mixtures thereof.

The ingestible compositions can, in certain embodiments, comprise any additional ingredients or combination of ingredients as are commonly used in food and beverage products, including, but not limited to:
acids, including, for example citric acid, phosphoric acid, ascorbic acid, sodium acid sulfate, lactic acid, or tartaric acid;
bitter ingredients, including, for example caffeine, quinine, green tea, catechins, polyphenols, green robusta coffee extract, green coffee extract, potassium chloride, menthol, or proteins (such as proteins and protein isolates derived from plants, algae, or fungi);
coloring agents, including, for example caramel color, Red #40, Yellow #5, Yellow #6, Blue #1, Red #3, purple carrot, black carrot juice, purple sweet potato, vegetable juice, fruit juice, beta carotene, turmeric curcumin, or titanium dioxide;
preservatives, including, for example sodium benzoate, potassium benzoate, potassium sorbate, sodium metabisulfate, sorbic acid, or benzoic acid;
antioxidants including, for example ascorbic acid, calcium disodium EDTA, alpha tocopherols, mixed tocopherols, rosemary extract, grape seed extract, resveratrol, or sodium hexametaphosphate;
vitamins or functional ingredients including, for example resveratrol, Co-Q10, omega 3 fatty acids, theanine, choline chloride (citocoline), fibersol, inulin (chicory root), taurine, Panax ginseng extract, guanana extract, ginger extract, L-phenylalanine, L-carnitine, L-tartrate, D-glucoronolactone, inositol, bioflavonoids, Echinacea, Ginko biloba, yerba mate, flax seed oil, garcinia cambogia rind extract, white tea extract, ribose, milk thistle extract, grape seed extract, pyrodixine HCl (vitamin B6), cyanoobalamin (vitamin B12), niacinamide (vitamin B3), biotin, calcium lactate, calcium pantothenate (pantothenic acid), calcium phosphate, calcium carbonate, chromium chloride, chromium polynicotinate, cupric sulfate, folic acid, ferric pyrophosphate, iron, magnesium lactate, magnesium carbonate, magnesium sulfate, monopotassium phosphate, monosodium phosphate, phosphorus, potassium iodide, potassium phosphate, riboflavin, sodium sulfate, sodium gluconate, sodium polyphosphate, sodium bicarbonate, thiamine mononitrate, vitamin D3, vitamin A palmitate, zinc gluconate, zinc lactate, or zinc sulphate;
clouding agents, including, for example ester gun, brominated vegetable oil (BVO), or sucrose acetate isobutyrate (SAIB);
buffers, including, for example sodium citrate, potassium citrate, or salt;
propylene glycol, ethyl alcohol, glycerine, gum Arabic (gum acacia), modified corn starch, silicon dioxide, magnesium carbonate, or tricalcium phosphate; or
starches and stabilizers, including, for example, polysorbate 60, polysorbate 80, medium chain triglycerides, and the like.

In some embodiments, component (a) can further comprise galact-oligosaccharides, fructo-oligosaccharides, acacia fiber, soluble pea fiber, soluble wheat fiber, arabinoxylan, isomalto-oligosaccharides, xylo-oligosaccharides, and the like.

The comestible composition can contain any of a number of ingredients, such as ingredients typically included in meat analogue products.

For example, in some embodiments, the comestible composition comprises a flavored water-in-oil emulsion according to any of the embodiments set forth in PCT Publication No. WO 2020/260628, which is hereby incorporated by reference.

In some embodiments, the comestible composition comprises encapsulated flavor compositions according to any of the embodiments set forth in PCT Publication No. WO 2021/104846, which is hereby incorporated by reference.

In some embodiments, the ingestible composition further comprises a carrier and, optionally, at least one adjuvant. The term “carrier” denotes a usually inactive accessory substance, such as solvents, binders, bulking agents, or other inert medium, which is used in combination with the present compound and one or more optional adjuvants to form the formulation. For example, water or starch can be a carrier for a flavored product. In some embodiments, the carrier is the same as the diluting medium for reconstituting the flavored product; and in other embodiments, the carrier is different from the diluting medium. The term “carrier” as used herein includes, but is not limited to, comestibly acceptable carrier.

The term “adjuvant” denotes an additive which supplements, stabilizes, maintains, or enhances the intended function or effectiveness of the active ingredient, such as the compound of the present invention. In one embodiment, the at least one adjuvant comprises one or more flavoring agents. The flavoring agent may be of any flavor known to one skilled in the art or consumers, such as the flavor of chocolate, coffee, tea, mocha, French vanilla, peanut butter, chai, or combinations thereof. In another embodiment, the at least one adjuvant comprises one or more ingredients selected from the group consisting of a emulsifier, a stabilizer, an antimicrobial preservative, an antioxidant, vitamins, minerals, fats, starches, protein concentrates and isolates, salts, and combinations thereof. Examples of emulsifiers, stabilizers, antimicrobial preservatives, antioxidants, vitamins, minerals, fats, starches, protein concentrates and isolates, and salts are described in U.S. Pat. No. 6,468,576, the content of which is hereby incorporated by reference in its entirety for all purposes.

The ingestible composition may further comprise a freezing point depressant, nucleating agent, or both as the at least one adjuvant. The freezing point depressant is an ingestibly acceptable compound or agent which can depress the freezing point of a liquid or solvent to which the compound or agent is added. That is, a liquid or solution containing the freezing point depressant has a lower freezing point than the liquid or solvent without the freezing point depressant. In addition to depress the onset freezing point, the freezing point depressant may also lower the water activity of the flavored product. The examples of the freezing point depressant include, but are not limited to, carbohydrates, oils, ethyl alcohol, polyol, e.g., glycerol, and combinations thereof. The nucleating agent denotes an ingestibly acceptable compound or agent which is able to facilitate nucleation. The presence of nucleating agent in the flavored product can improve the mouthfeel of the frozen Blushes of a frozen slush and to help maintain the physical properties and performance of the slush at freezing temperatures by increasing the number of desirable ice crystallization centers. Examples of nucleating agents include, but are not limited to, calcium silicate, calcium carbonate, titanium dioxide, and combinations thereof.

In some embodiments, the ingestible composition is formulated to have a low water activity for extended shelf life. Water activity is the ratio of the vapor pressure of water in a formulation to the vapor pressure of pure water at the same temperature. In one embodiment, the ingestible composition has a water activity of less than about 0.85. In another embodiment, the ingestible composition has a water activity of less than about 0.80. In another embodiment, the ingestible composition has a water activity of less than about 0.75.

### Methods of Preparation

The ingestible compositions disclosed herein can be made by any suitable means, as typically employed in the manufacturing of products containing fiber blends. Such methods include dry mixing, granulating, encapsulating, spray drying, and the like. In some embodiments, the ingestible compositions are prepared by spray drying, where the protein compositions are mixed with a liquid medium that is removed by drying during a spraying process. Spray drying processes are well known in the art of preparing comestible products. Any suitable spray-drying process can be used and optimized for the components used in the ingestible compositions. In some embodiments, the ingestible compositions are prepared by extrusion, where the protein compositions are mixed with other materials and is formed into an shaped article by extrusion. Extrusion processes are well known in the art of preparing comestible products. Any suitable extrusion process or apparatus can be used and optimized for the components used in the ingestible compositions.

### Flavored Products

In certain aspects, the disclosure provides a flavored product, which comprises the ingestible composition according to any of the embodiments set forth above. In some embodiments, the flavored product is a food product, such as a meat analogue product, for example, a non-animal-based ground beef replica. In some other embodiments, the flavored product is an animal feed product, such as pet food product. In such flavored products, the comestible composition can, in some embodiments, be used in combination with animal-based products to reduce the degree of animal fats or animal products in the comestible product. In other embodiments, the flavored products contain no animal-based products, such that the comestible composition is used to make an analogue or a replica of a meat product, such as a ground beef patty.

In some other embodiments, the flavored product is a meat-replacement product (or meat analogue), such as a product designed to mimic products traditionally made from red meat. For example, the flavored product can be a meat analogue dough, such as those described in PCT Publication No. WO 2015/153666. Such flavored products can be designed to simulate beef products, such as ground beef (for making burgers) or cuts of beef for inclusion in soups, prepared meals, and the like. The flavored products can also be designed to simulate cuts or ground forms of other meat, such as chicken, turkey, pork, goat, lamb, venison, and bison, or seafood, such as fish or shellfish (crab, scallop, shrimp, squid, and the like).

### EXAMPLES

To further illustrate this invention, the following examples are included. The examples should not, of course, be construed as specifically limiting the invention. Variations of these examples within the scope of the claims are within the purview of one skilled in the art and are considered to fall within the scope of the invention as described and claimed herein. The reader will recognize that the skilled artisan, armed with the present disclosure, and skill in the art is able to prepare and use the invention without exhaustive examples.

### Example 1—Pea Protein and Pectin Complex

Pea protein isolate was introduced to demineralized water at a concentration of 5% (w/w) giving a soluble protein content in the supernatant of 1.28% (w/w) at pH 3.5. The composition was left to hydrate for 2 hours under agitation at 400 rpm. Low-methoxyl (LM) pectin was separately introduced to demineralized water at a concentration of 0.5% (w/w) and was stirred for 2 hours at 400 rpm. After 2 hours of hydration, the protein solution was adjusted to pH 3.5 with anhydrous citric acid and left to equilibrate for 30 minutes. The whole solution was transferred to centrifugation tubes and centrifugated during 15 minutes at 4000 rpm. The supernatant was withdrawn und put aside for precipitation formation. The pectin solution was adjusted to pH 3.5 with 0.1 M HCl and left to equilibrate for 30 minutes.

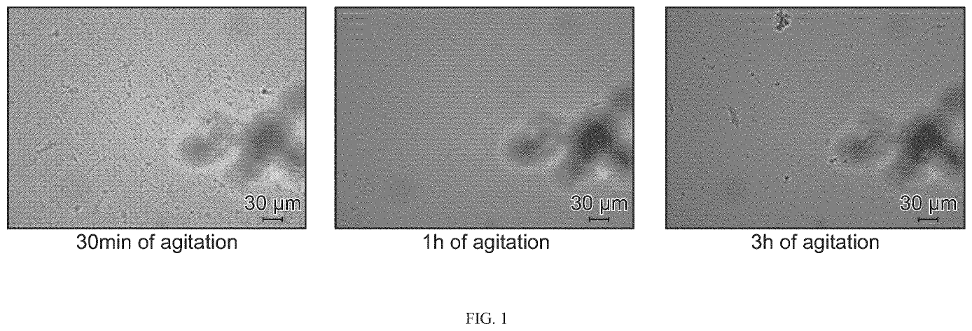

Precipitates were formed at pH 3.5 and a weight ratio of 2.6:1 (plant protein/pectin) and a total biopolymer concentration of 0.37 wt % (0.27 wt % of pea protein and 0.1 wt % of LM pectin). At this ratio the measured Zeta potential of the precipitates was −6.5 mV. FIG. 1 shows the precipitate formation at 30 minutes, 1 hour, and 3 hours, with the unit of measurement being 30 μm. The maximum particle size of the precipitates was measured to be about 30 μm.

The precipitates were recovered and a sensory evaluation of their off-note masking potential was carried out. Five panelists tested the precipitates in solution in comparison to a reference sample of pea protein solution in demineralized water at 0.27% (w/w) adjusted with anhydrous citric acid to pH 3.5. The sensory panelists noted that the precipitates exhibited a less pronounced intensity of the off-note descriptors of nutty, cereal, and beany.

### Example 2—Pea Protein and Gum Arabic

Pea protein/gum Arabic precipitates were formed at pH 3.5 and a weight ratio of 3.6:1. The total biopolymer concentration was 1.15 wt % (0.9 wt % pea protein isolate/0.25 wt % Gum Arabic). The pea protein composition was formed in the same way as described in Example 1. The gum Arabic mother composition contained 2.25 wt % of gum Arabic in demineralized water. The pH was adjusted to a pH of 3.5 using 0.1M HCl. The pea protein composition and the gum Arabic composition were mixed at the above-mentioned ratio followed by stirring for 2 hours.

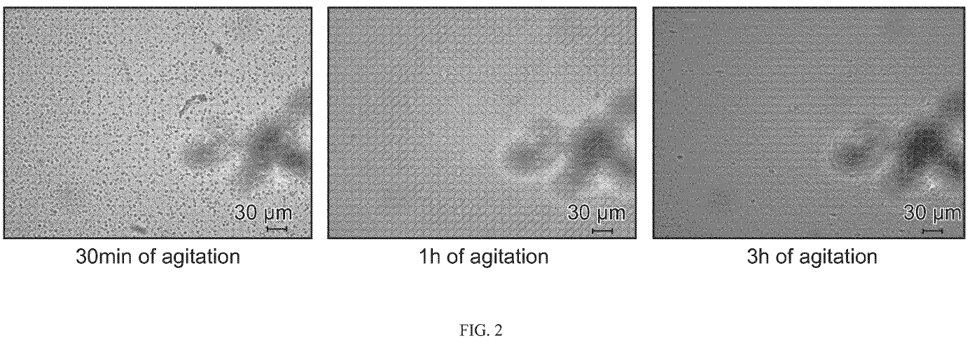

FIG. 2 shows the precipitate formation at 30 minutes, 1 hour, and 3 hours, with the unit of measurement being 30 μm.

The precipitates were recovered and a sensory evaluation of their off-note masking potential was carried out. Five panelists tested the precipitates in solution in comparison to a reference sample of pea protein solution in demineralized water at 0.9% (w/w) adjusted with anhydrous citric acid to pH 3.5. The sensory panelists noted that the precipitates exhibited a less pronounced intensity of the off-note descriptors of astringency and acidity.

### Example 3—Pea Protein and Carageenan

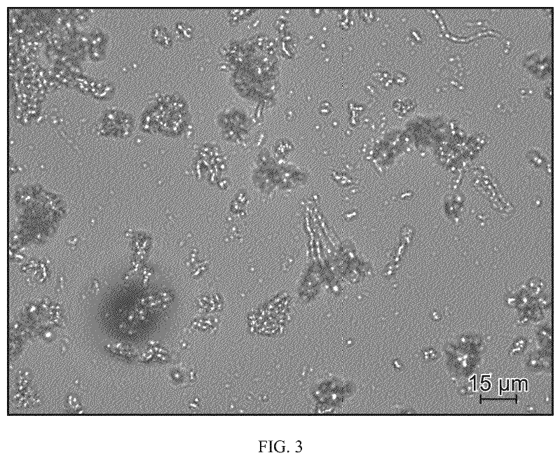

Pea protein/carrageenan precipitates were prepared using procedures analogous to those used in Examples 1 and 2. Pea protein isolate at 5% (w/w) was brought to pH 3 with HCl and a carrageenan (SATIAGUM, Cargill, Wayzata, MN) solution at 1% (w/w) was adjusted to pH 3 with HCl. The supernatant with the soluble protein share was mixed at a ratio 2.8:1 with the carrageenan solution and completed with demineralized water at pH 3. The blend was stirred for 2 hours. The formed precipitates were characterized by an average Zeta potential of −2.9 mV. The complexation yield was calculated to be 68%. FIG. 3 shows the precipitate formed, with the unit of measurement being 15 μm.

### Example 4—Pea Protein, Gum Arabic, and Carrageenan

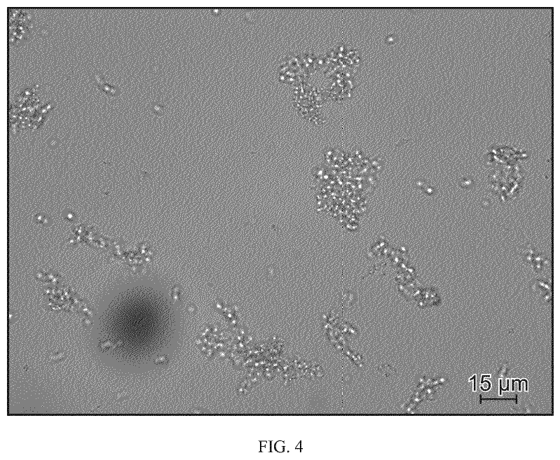

The following solutions were prepared: (a) pea protein isolate solution at 5% (w/w) was brought to pH 3 with HCl, (b) carrageenan (SATIAGUM, Cargill, Wayzata, MN) at 1% (w/w) was brought to pH 3 with HCl, and (c) a 2.5 wt % gum Arabic solution was adjusted to pH 3 with HCl. First the carrageenan and gum Arabic solutions were diluted and mixed together at a concentration of 0.1 wt % for the carrageenan solution and 0.21 wt % for the gum Arabic solution, respectively. This blend was stirred for 15 minutes at 400 rpm. Subsequently, the pea protein solution was added slowly upon further stirring and completed to 100% with demineralized water at pH 3. The stirring of the solution was continued for another 2 hours. The weight ration of pea protein to gum Arabic to carrageenan was 9.7:2.1:1. The complexation yield was calculated to be 50%, and the average Zeta potential of the formed precipitates was 5.45 mV. All precipitates show stability upon a wide range of pH (i.e., from 3.5 to 6.6). FIG. 4 shows the precipitate formed, with the unit of measurement being 15 μm.

### Example 5—Pea Protein and Gum Arabic with Spray Drying

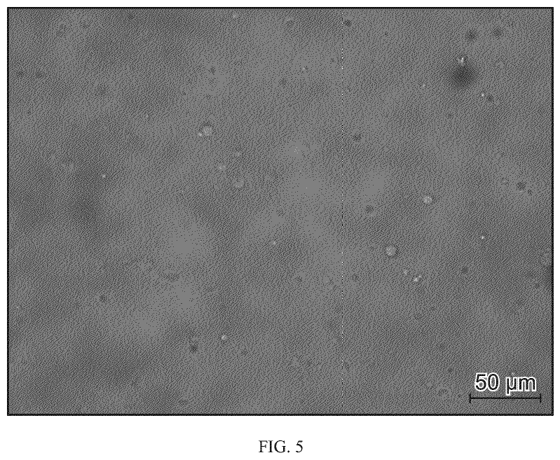

Two solutions were prepared: (a) a 5% (w/w) pea protein isolate and a 1.3% (w/w) gum Arabic solution. After hydration of the pea protein solution, the pH of both solutions was adjusted to pH 3.6. The pea protein isolate was positively charged and interacted quickly with the anionic gum Arabic. The protein solution was centrifugated and only the supernatant was used, with a determined soluble protein content of 1.75% (w/w). The mixing ratio was carried out at pea protein/gum Arabic weight ratio of 3:1. The total biopolymer concentration was around 1.4% (w/w) before addition of the carrier, which was Maltodextrin 18 DE, and was included in a total concentration of 25% (w/w). The total blend was left to agitate for 2 hours and stored in the refrigerator overnight. The mix was spray dried the following day using standard spray drying techniques. The formed coacervates survive the spray drying process, as shown in FIG. 5.

### Example 6—Pea Protein and Sunflower Protein

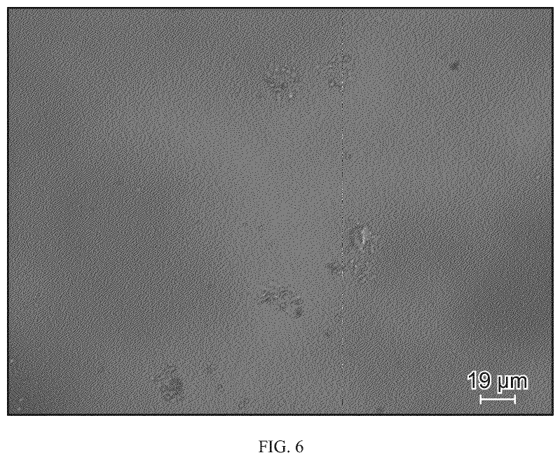

A 5% (w/w) pea protein isolate solution and a 2% (w/w) sunflower protein solution were prepared and left to hydrate overnight. The pH of both solutions was adjusted to pH 3.5 and the supernatant was withdrawn after 15 minutes of centrifugation at 4000 rpm. The solutions were mixed at a sunflower protein/pea protein weight ratio of 2:1 and agitated for 2 hours to form precipitates. Once the precipitates are formed, the carrier was introduced. The carrier was Maltodextrin 18DE and was added and the total. After two hours of agitation the blend was spray dried. The formed precipitates survived the drying process, as shown in FIG. 6.

### Example 7—Pea Protein and Canola Protein

A pea protein isolate solution was prepared 5% (w/w), agitated for 2 hours and left to hydrate in the refrigerator overnight. A canola protein solution was prepared 2% (w/w), agitated for 2 hours, and left to hydrate in the refrigerator overnight. Samples were left at pH native of around pH 7.3 for pea protein isolate, around pH 6.3 for canola protein. The samples were centrifugated (@4500 rpm for 15 minutes) and the supernatant was withdrawn (with soluble protein for pea protein isolate of 2.6%, −25 mV, and 1.8% soluble protein, +4 mV, for canola protein). The samples were then mixed at a ratio of 1.4:1 with total final biopolymer concentration of around 2% (w/w). The precipitates formed instantaneously. The sample was left to agitate overnight and the sample was analyzed by microscopy.

### Example 8—Algae Protein and Wheat Gluten

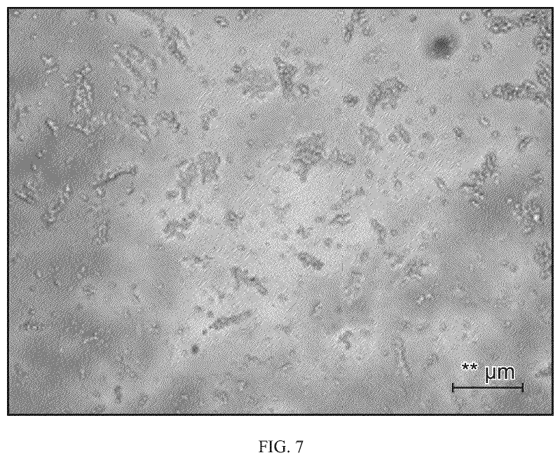

A chlorella algae protein solution was prepared at 5% (w/w) and was agitated for 2 hours and left to hydrate in the refrigerator overnight. A wheat gluten solution was prepared 2% (w/w), and agitated for 2 hours, and left to hydrate in the refrigerator overnight. Samples were left at pH native, around pH 5.6 for chlorella protein, and around pH 4.9 for wheat gluten. The Zeta potential is −11 mV for chlorella protein at pH nat and at +9 mV for wheat gluten. The samples were centrifugated (@4500 rpm for 15 minutes) and the supernatant was withdrawn. The soluble protein in the chlorella protein sample is at 0.7% (w/w) whereas the that of the wheat gluten is at 1.6% (w/w). The samples were then mixed at a ratio of 2.2:1 with total final biopolymer concentration of around 0.76% (w/w). The precipitates formed quickly. FIG. 7 shows a micrograph of the precipitates.

### Example 9—Wheat Protein and Potato Gluten

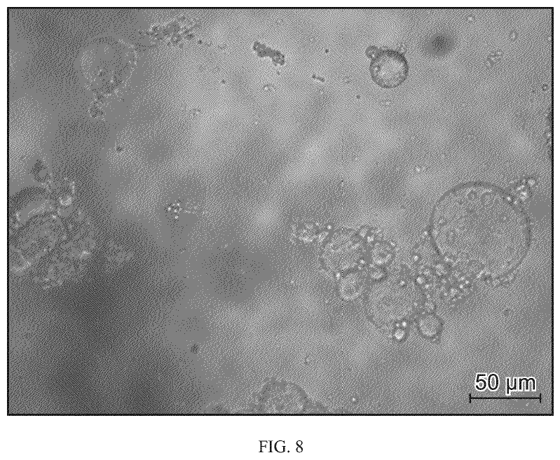

A wheat protein solution at 2% protein content was emulsified with a 10-times higher amount of oil (capric/caprylic triglyceride). A potato protein supernatant was added under stirring to sum up to a total biopolymer concentration of 0.8% (R=1). The whole process was executed at about pH 5.5. After 2 hours of stirring, transglutaminase was added (40 U/g of protein). The dispersion was heated to 40° C. and stirred for 2 hours and continued to stir overnight at room temperature. The enzyme was deactivated at 80° C. for at least 30 minutes. The wheat protein adsorbed on the surface of the particle membrane which was rigid and retracted after squeezing. FIG. 8 shows a micrograph of the microparticles after enzyme deactivation.

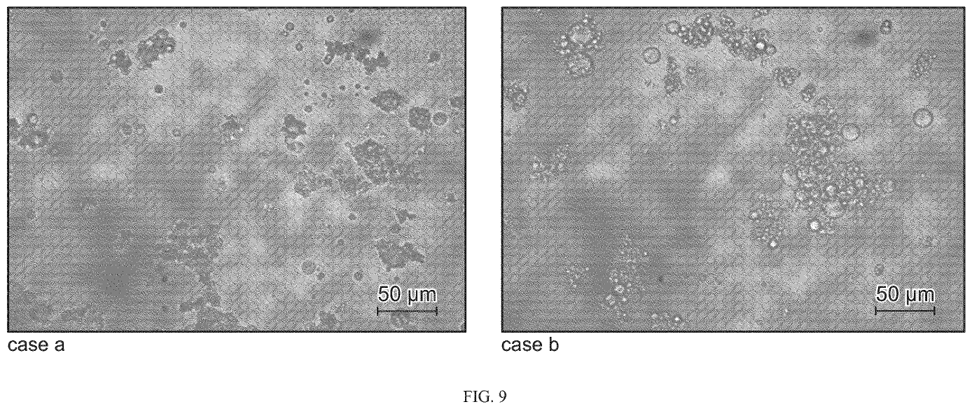

The same protocol was used as described above, except that the oil concentration was lowered to 1% (case a) and 1.66% (case b). FIG. 9 shows micrographs of the microparticles formed after inactivation step at magnification 40×, with case a on the left and case b on the right.

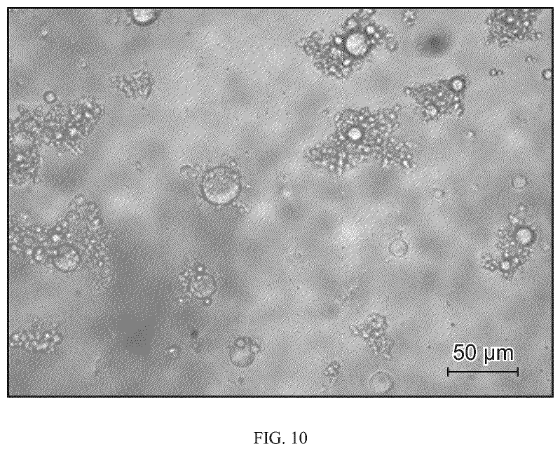

Both proteins were mixed at pH 2 to avoid electrostatic interaction; the emulsion with the added secondary protein is then adjusted step by step to a final pH of 5.35 and left to stir at room temperature for 2 hours. Tgase was added, holding the temperature at 40° C. for three hours followed by inactivation at 80° C. for 30 minutes. FIG. 10 shows a micrograph is taken after inactivation, magnification 40×.

### Example 10—Canola Protein and Potato Protein

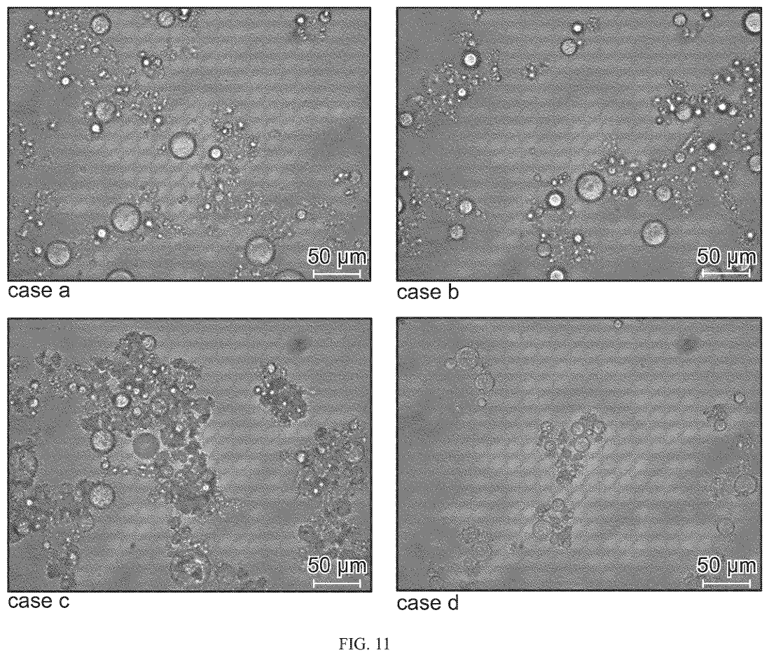

A potato protein was used to emulsify a final concentration of 10% of oil (capric/caprylic glyceride). Canola protein concentrate was then added to sum up to a final protein concentration of 4% with a relative weight ratio of R_{Canola/Potato}=3 (case a), 2 (case b), 1 (case c), 0.5 (case d). The mixtures were stirred at room temperature followed bt Tgase addition, holding the temperature at 40° C. for 3 hours. Then the enzyme was inactivated at 80° C. for 30 minutes. FIG. 11 shows micrographs at 40× magnification for the microparticles formed for each of the four cases after inactivation.

### Example 11—Sensory Testing

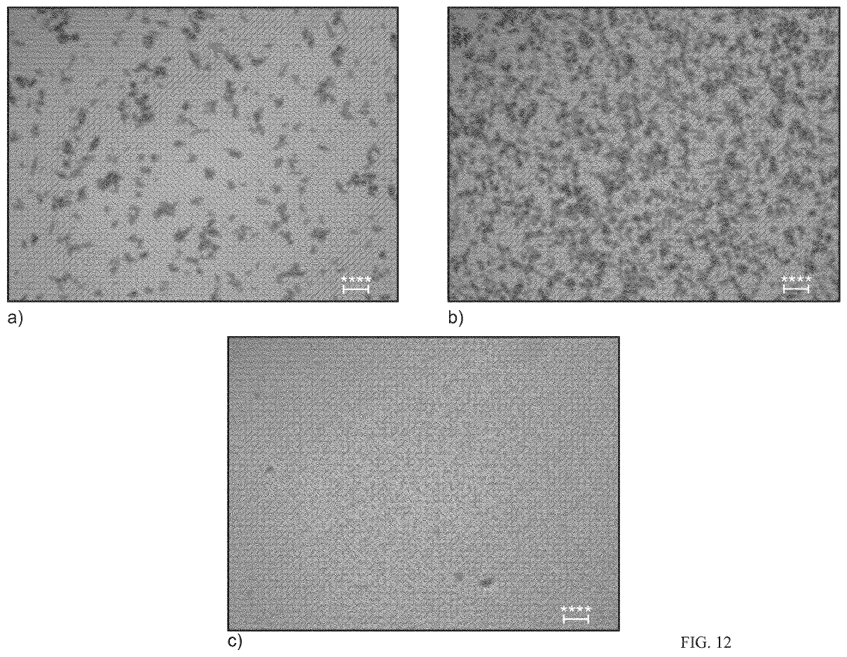

Plant protein solutions were prepared in demineralized water with pea protein isolate at a concentration of 5%. A second plant protein solution was prepared, where the second protein is chosen either from a plant protein isolate, such as canola and potato protein, and or from protein concentrate, such as soluble wheat protein, at 2%. The solutions were hydrated for 2 hours at room temperature. The pH was adjusted to pH 6 with 1M and 0.1M food grade HCl for pea protein isolate and canola protein isolate, respectively, and with NaOH for potato and wheat protein. The solutions were kept at magnetic stirring for 2 hours. The total solutions were centrifugated at 4500 rpm for 15 minutes and the supernatant was withdrawn subsequently and analyzed gravimetrically by TGA. The ionic charge was measured by Zetasizer (Malvern). Pea protein isolate, canola protein isolate, potato protein isolate, and wheat protein contain a soluble protein part of 1.61%, 1.89%, 1.19% and 1.18%, respectively. Whereas pea protein isolate is charged positively, the second protein is characterized by a negative charge. Complexes were formed at a ratio of 1:1 for pea protein isolate and canola or potato protein isolate and at 1:2 for the combination with wheat protein. The solutions were blended to obtain a maximal total biopolymer concentration and left for agitation during 30 min. FIG. 12 shows micrographs of the formed complexes a) pea protein isolate/canola protein isolate; b) pea protein isolate/potato protein isolate; c) pea protein isolate/soluble wheat gluten).

Half of each solution was kept in this state, and the other half was pasteurized at 92° C. for 10 minutes. The solutions were kept in the fridge overnight, together with both protein isolate supernatants.

Sensory evaluation was performed on cleaned slurry diluted to 0.5% of total biopolymer (0.25% pea protein isolate and 0.25% second protein). Cleaning was done by discarding the supernatant of the complex solution and re-dilution of the complexes by addition of demineralized water. Pea protein isolate supernatant diluted to 0.25% as reference sample to evaluate off-note masking of the formed complexes. Table 1 shows the results of sensory evaluation, where about 20 panelists evaluated each of the qualities on a scale of 0 to 10, with 10 indicating greatest intensity. Sensory evaluation of mixtures and individual components. Reference being 0.25% solution of pea protein isolate; complexes formed by complexation of pea protein isolate with a second plant protein at total biopolymer concentration of 0.5% (according to theoretical pea protein isolate concentration of 0.25%).

TABLE 1

| | Pea (Ref) | Pea/Wheat | Pea/Potato | Pea/Canola |
|---|---|---|---|---|
| Overall intensity | 4.9 | 5.1 | 4.9 | 5.6 |
| Whole cereal note | 5.6 | 4.2 | 2.4 | 3.0 |
| Green vegetal note | 2.6 | 2.2 | 1.9 | 2.3 |
| Sourness | 1.1 | 0.8 | 1.2 | 2.1 |
| Bitterness | 2.9 | 3.8 | 3.2 | 3.8 |
| Astringency | 2.3 | 3.0 | 5.3 | 4.3 |
| Sweetness | 0.9 | 1.3 | 1.1 | 2.0 |
| Thickness | 2.3 | 3.8 | 3.1 | 4.1 |
| Mouthcoating | 3.0 | 3.7 | 3.4 | 3.7 |

It was observed that the intensity of negatively perceived pea protein descriptors such as “whole cereal” and “green vegetable” of the complexes is reduced in comparison to the reference sample. The complexes are enhancing the texture attributes of “thickness” of the evaluated sample and therefore are demonstrated to have an effect on texture.

### Example 12—Sensory Testing

Complexes of pea protein and canola protein were prepared for evaluation by representative panel of about 20 panelists. Sample preparation is the same as in Example 11. For sensory evaluation the reference solutions, being pasteurized pea protein isolate supernatants diluted to final concentration of 0.5% and 0.25%, respectively, non-pasteurized pea protein and canola protein isolate supernatant solutions at 0.5% and complexes in slurry or in the cleaned version. Cleaning consisted of discarding the excess protein in the supernatant of the formed complexes followed by re-dilution with demineralized water to aimed final concentration.

Nine descriptors were rated on an intensity scale of 0 to 10 (not intense to very intense). Table 2 summarizes the results.

TABLE 2

| | | | | | | Canola |
|---|---|---|---|---|---|---|
| | | | | | Pea (0.5%) | (0.5%) |
| | Complex | Complex | Pea (0.5%) | Pea (0.25%) | Not | Not |
| | Slurry | Cleaned | Pasteurized | Pasteurized | Pasteurized | Pasteurized |
| Overall | 5.6 | 3.5 | 3.9 | 3.6 | 4.0 | 5.0 |
| intensity | | | | | | |
| Whole cereal | 4.9 | 3.3 | 4.3 | 3.5 | 3.8 | 2.9 |
| note | | | | | | |
| Green vegetal | 3.3 | 1.8 | 2.8 | 3.0 | 3.3 | 2.1 |
| note | | | | | | |
| Sourness | 0.6 | 0.7 | 0.6 | 0.4 | 0.7 | 2.3 |
| Bitterness | 2.7 | 3.0 | 2.3 | 2.1 | 3.0 | 3.9 |
| Astringency | 4.0 | 3.3 | 3.0 | 3.1 | 2.9 | 4.5 |
| Sweetness | 1.3 | 0.6 | 0.5 | 0.7 | 0.6 | 2.1 |
| Thickness | 3.2 | 2.3 | 1.9 | 1.3 | 2.0 | 1.6 |
| Mouthcoating | 4.9 | 2.5 | 2.1 | 1.9 | 2.2 | 2.2 |

These results demonstrate how the formulations provide a reduction of the “whole grain” and “green vegetable” descriptor intensity and at the same time enhance texture attributes such as “mouthcoating” and “thickness”.

### Example 13—Emulsions

A solution of 2% w/w potato protein isolate solution was prepared, and an oil-based flavor composition was emulsified with the protein solution using an rotor/stator homogenizer at a speed of 10000 rpm. A wheat protein supernatant (as in Example 11) was added under magnetic stirring to sum up to a total biopolymer concentration of 0.8% (R=1). The process was performed around pH 5.5. The emulsion was then left to stir for two hours and placed in the refrigerator. It was observed that the wheat protein adsorbed on the surface of the particle membrane enhancing the rigidity of droplet membrane. Similar examples were prepared using an oil perfume composition instead of a flavor composition.

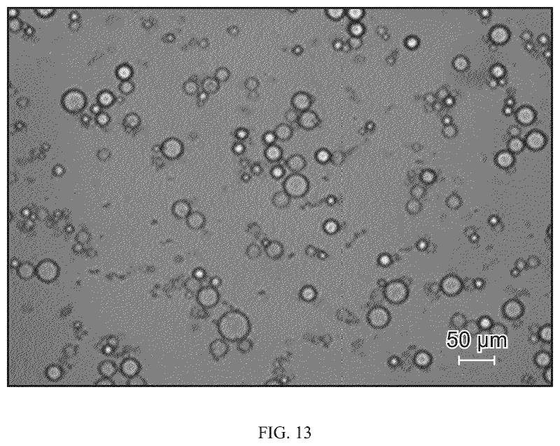

Potato protein isolate at 2% was used to emulsify a final concentration of 10% of oil (capric/caprylic glyceride). Canola protein isolate solution at 2% was added to sum up to a final protein concentration of 4% with mass ratios of R_{Canola/Potato} of 3, 2, 1, and 0.5. Canola protein concentrate absorbed onto the membrane of the oil emulsified with a potato protein isolate. The protein ratio influenced the properties of the membrane. For the combination used here, ratios of R=3 and R=2 provided a membrane with enhanced rigidity, as identified by visual assessment of the membrane deformation behavior by compression between two glass slides with brightfield microscopy. This ratio can therefore be used to finetune the properties of the membrane, to obtain softer or more rigid membranes. FIG. 13 shows a micrograph of the formed particles, where the lower bar represents 50 microns.

### Example 14—Protein/Protein Paste for Off-Note Masking

The opposite charges of pea protein and canola protein isolate at the native pH of both (being 7.5 and pH 6, respectively) were used advantageously here. Pea protein isolate and canola protein powder were mixed in a kitchen blender assuming a solubility of 1 g soluble for 5 g and 1 g for 1 g, respectively. Water was slowly added to the dry mix; the blend was kneaded for 30 minutes. The end-product was a humid paste at a 40/60 or 50/50 protein/water content. Protein interaction was evaluated visually by microscopy. The produced paste was extruded. The favored protein/protein interaction is believed to lead to a blocking of the binding sites responsible for the pronounced off-notes and therefore reducing the negatively perceived descriptors such as “whole grain” and “green vegetable”. The particle size of the formed structures was up to 50 micrometers.

### Example 15—Spray-Dried Complexes & Dairy Analogues

Complexes were as formed in Example 11 and spray dried using maltodextrin as carrier (at a concentration of approximately 20%). The spray dried powder was i) blended with pea protein isolate powder before extrusion at R=1 or used in totality for high moisture extrusion. The final product had reduced off-notes compared to an extrudate purely produced with pea protein isolate; ii) can be used as basis for fermented dairy analog products with reduced off-notes; and iii) was diluted with water for a final protein concentration of 3 or 6%, the spray dried particles are stabilized with a clean label colloidal stabilizer.

### Example 16—Gelled Droplets and Mouthfeel Improvement for Plant-Based Burger

Pea protein isolate and sunflower protein solutions were prepared at a concentration of 5%. The solutions were stirred for 2 hours to allow for total hydration. Subsequently, the pH was adjusted with food grade HCl to pH 3; the solutions underwent stirring during additional 2 hours time. The protein solutions were centrifugated for 15 minutes at 4500 rpm and the supernatant was withdrawn. Meanwhile a carrageenan solution was prepared at a final concentration of 1%. The carrageenan can be kappa or iota or a mixture of these, preferably iota. Kappa carrageenan improves gel strength while iota carrageenan improves flexibility.

To prepare the gelled droplets, the carrageenan solution was added to the pea protein isolate supernatant under agitation using a pipette (Step A). The formed droplets were stirred at room temperature for 15 min to allow formation of stable droplet membranes. The second protein solution was added with subsequent stirring during 15 minutes. Transglutaminase was added to the solution. The activation and de-activation were performed at 40° C. for 2 hours and 80° C. for 30 minutes, respectively. The gelled droplets were sieved and transferred to demineralized water where they keep their shape and rigidity. The produced prototype can be incorporated into meat analog beef patties for enhanced mouthfeel. The droplets were characterized by a particle size around 5 mm and are therefore visible by the naked eye.

Alternatively, Step A (addition of the carrageenan solution) can be performed using various dosing techniques, including (i) via a capillary connected to a tube and syringe, with the dosing performed by a syringe pump (ii) using prilling/jetting device, or (iii) using a spray nozzle if smaller drops are desired. Smaller droplets can be created by dosing the carrageenan solution from a smaller diameter orifice, and larger droplets can be created by using a larger diameter orifice.

### Example 17—Cross-Linking

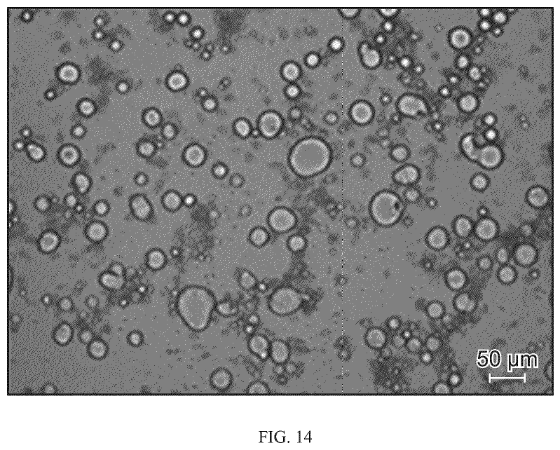

An emulsion was prepared according to Example 13. After completing all the steps described in Example 13, transglutaminase was added for crosslinking at a concentration of 0.3% w/w (per total mass of liquid). The dispersion was heated to 40° C. and stirred for 2 hours and continued to stir overnight at room temperature. The enzyme was deactivated at 80° C. for at least 30 minutes. The result was a slurry of soft capsules with crosslinked protein/protein membrane. FIG. 14 shows a micrograph of the formed emulsions, where the lower bar represents 50 microns.

### Example 18—Soft Capsule Processing

To obtain a dry powder of the formulations described in Examples 13 and 14, the emulsions from Example 13 and the soft capsules from Example 17 were further processed by spray-drying using a laboratory scale spray-dryer. Maltodextrin with dextrose equivalent DE 18 was added to 20% w/w in the water phase and the emulsions obtained in Example 13 or the soft capsules in Example 17 were processed to powdered form. (Spray drying parameter: flow rate 100 ml/hour, air temperature setting 190 deg C.).

### Example 19—Combination of Cross-Linked and Non-Cross-Linked

By mixing emulsions as prepared in Example 13, and slurries of soft capsules crosslinked according to Example 17, it is possible to obtain a combined system comprising a mixture of non-crosslinked emulsion drops and crosslinked soft capsules. Such mixtures may be beneficial if a flavor or other oil-based active needs to be delivered at different time scales, with the non-crosslinked oil drops allowing immediate release, while the crosslinked soft capsule drops provide extended release. Alternatively, different oil-based flavor compositions may be used for the non-crosslinked and crosslinked emulsions.

Such combined systems may also be spray-dried; combined dry systems may also be obtained by powder-blending the separate dried emulsion and the dried slurry of soft capsules after preparing and drying them individually.

It is understood that for any of the combinations described in this example, the emulsion or the slurry of soft capsules may comprise the same flavor or perfume composition, or each of them may comprise a different flavor or perfume compositions.

1. A polyelectrolyte complex comprising a polycationic non-animal protein and polyanionic polymer, wherein the polycationic non-animal protein and the polyanionic polymer are associated via electrostatic attraction, and wherein the polyanionic polymer comprises a plant protein.

2. The polyelectrolyte complex of claim 1, wherein the polycationic non-animal protein is a polycationic plant protein.

3. The polyelectrolyte complex of claim 1, wherein the polycationic non-animal protein is pea protein, soy protein, fava bean protein, potato protein, or a combination thereof.

4. The polyelectrolyte complex of claim 1, wherein the polyanionic polymer further comprises pectin, gum Arabic, carrageenan, or any combination thereof.

5. The polyelectrolyte complex of claim 1, wherein the polyanionic polymer comprises sunflower protein, potato protein, or a combination thereof.

6. The polyelectrolyte complex of claim 1, wherein the polyelectrolyte complex is in the form of a particle.

7. The polyelectrolyte complex of claim 6, wherein the particle is formed by precipitation from an aqueous medium.

8. The polyelectrolyte complex of claim 6, wherein the particle is formed by remove an aqueous medium, such as through spray drying.

9. An ingestible composition comprising a polyelectrolyte complex of claim 1.

10. The ingestible composition of claim 9, wherein the polyelectrolyte complex makes up from 1 wt % to 80 wt % of the ingestible composition, based on the total dry weight of the ingestible composition.

11. The ingestible composition of claim 9, further comprising a plant fiber.

12. A flavored product comprising an ingestible composition of claim 9.

13. The flavored product of claim 12, wherein the flavored product is a dairy analogue or a meat analogue.

14-15. (canceled)

16. A method of reducing one or more off notes of a polycationic non-animal protein, the method comprising forming a polyelectrolyte complex between the polycationic non-animal protein and a polyanionic polymer, wherein the polyanionic polymer comprises a plant protein.

17. A method of enhancing a mouthfeel or a perceived juiciness or perceived thickness of a polycationic non-animal protein, the method comprising forming a polyelectrolyte complex between the polycationic non-animal protein and a polyanionic polymer, wherein the polyanionic polymer comprises a plant protein.
